(19) United States
(12) Patent Application Publication
(10) Pub. No.: US 2025/0320238 A1
(43) Pub. Date: Oct. 16, 2025

(54) TRANSITION METAL CLUSTER COMPOUND, PHOTOSENSITIVE COMPOSITION, PATTERN FORMING METHOD, AND METHOD FOR PRODUCING SUBSTRATE

(71) Applicants: Mitsubishi Chemical Corporation, Tokyo (JP); National Institutes For Quantum Science And Technology, Chiba (JP)

(72) Inventors: Shigeki Hattori, Tokyo (JP); Kazuhiro Nagayama, Tokyo (JP); Akane Kato, Tokyo (JP); Keishi Yamamoto, Tokyo (JP); Hiroki Yamamoto, Chiba (JP)

(73) Assignees: Mitsubishi Chemical Corporation, Tokyo (JP); National Institutes For Quantum Science And Technology, Chiba (JP)

(21) Appl. No.: 19/250,422

(22) Filed: Jun. 26, 2025

Related U.S. Application Data
(63) Continuation of application No. PCT/JP2023/044190, filed on Dec. 11, 2023.

(30) Foreign Application Priority Data
Dec. 26, 2022 (JP) 2022-208686

(51) Int. Cl. C07F 7/28; G03F 7/004; G03F 7/20

(52) U.S. Cl. CPC C07F 7/28; G03F 7/0042; G03F 7/2016

(57) ABSTRACT

A transition metal cluster compound is capable of realizing fine circuit patterns, a photosensitive composition contains the transition metal cluster compound, and a pattern forming method uses the photosensitive composition. The transition metal cluster compound contains transition metal elements and a ligand represented by the following general formula (1):

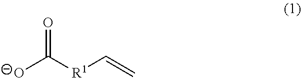

wherein R¹ is a hydrocarbon chain having one or more carbon atom.

### CROSS-REFERENCE TO RELATED APPLICATIONS

This patent application is a bypass continuation of International Application No. PCT/JP2023/044190, filed on Dec. 11, 2023, and claims the benefit of priority to Japanese Application No. 2022-208686, filed on Dec. 26, 2022. The content of each of these applications is hereby incorporated by reference in its entirety.

### BACKGROUND OF THE INVENTION

### Field of the Invention

The present invention relates to a transition metal cluster compound suitable for use in ultra-microlithography processes, such as the production of ultra-LSIs and high-capacity microchips, and other photofabrication processes, a photosensitive composition containing the same, and a pattern forming method using the photosensitive composition.

### Description of Related Art

Conventionally, in the production process of semiconductor devices, lithography fine processing is performed by using photoresist compositions.

In semiconductor photolithography, in accordance with Moore's Law, circuit patterns become smaller as semiconductor devices become more miniaturized, and further miniaturization is desired.

Photolithography is broadly based on the shortening of wavelengths of light sources in exposure devices and the use of photoresists that respond to this. Photoresists are required to meet all of the requirements of high resolution, low roughness, and high sensitivity. Conventional resists that contain photoacid generators, known as chemically amplified resists, cause a decrease in resolution due to diffusion of acid, and are therefore considered incapable of handling ultra-fine patterns.

In recent years, non-chemically amplified photoresists based on compounds containing metal elements such as Zn and Sn have been proposed, and it has been reported that they can be used to form fine patterns using next-generation exposure devices that use extreme ultraviolet (EUV) rays.

For example, Patent Literatures 1 to 5 and Non-Patent Literatures 1 to 3 listed below disclose methods of forming resist patterns using extreme ultraviolet (EUV) rays or electron beams.

### CITATION LIST

### Patent Literature

Patent Literature 1: Japanese Patent Laid-Open No. 2015-108781
Patent Literature 2: Japanese Patent Laid-Open No. 2001-072716
Patent Literature 3: Japanese Patent Laid-Open No. 2017-173537
Patent Literature 4: Japanese Patent Laid-Open No. 2012-185484
Patent Literature 5: Japanese Patent Laid-Open No. 2021-102604

### Non-Patent Literature

Non-Patent Literature 1: Minoru Toriumi etc., Proc. SPIE, 9779 (2016) 97790G
Non-Patent Literature 2: Lianjia Wu etc., Proc. SPIE, 10957 (2019) 109570B
Non-Patent Literature 3: Neha Thakur etc., Proc. SPIE, 10957 (2019) 10957D

### SUMMARY OF THE INVENTION

### Problem to be Solved by the Invention

In the field of photolithography, especially semiconductor photolithography, there is a need for photosensitive compositions and pattern forming methods that can achieve further miniaturization of circuit patterns.

The present inventors have found, as a result of intensive study, that a metal cluster compound composed of a carboxylic acid containing an alkyl or aryl chain that is easily dissolved in an organic solvent before polymerization, which has a vinyl group terminal that is easily polymerized, and a metal oxide, is suitable as a photoresist for handling ultra-fine patterns.

Therefore, an object of the present invention is to provide a transition metal cluster compound capable of achieving miniaturization of circuit patterns, a photosensitive composition containing the same, and a pattern forming method using the photosensitive composition.

### DETAILED DESCRIPTION OF THE INVENTION

The present invention has the following aspects.

[1]A transition metal cluster compound comprising a transition metal element and a ligand represented by the following general formula (1), wherein the compound forms a cluster centered on the transition metal element:

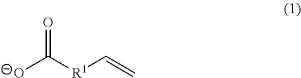

wherein R¹ is a hydrocarbon chain having one or more carbon atoms.

[2] The transition metal cluster compound according to [1], wherein the transition metal element comprises at least one element selected from the group consisting of zirconium, hafnium, and titanium.

[3] The transition metal cluster compound according to [1] or [2], comprising 2 or more and 12 or less of the transition metal elements and a ligand represented by the general formula (1).

[4] The transition metal cluster compound according to any one of [1] to [3], wherein R¹ is a saturated hydrocarbon chain having one or more carbon atoms.

[5] The transition metal cluster compound according to any one of [1] to [4], wherein the ligand is a ligand represented by the following general formula (2) or (3):

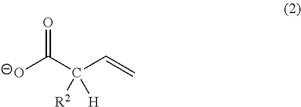

wherein R² is a hydrocarbon group; and

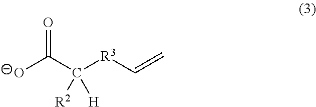

wherein R² is a hydrocarbon group, and R³ is a hydrocarbon chain having one or more carbon atoms.

[6] The transition metal cluster compound according to [5], wherein R² has 1 to 6 carbon atoms.

[7] The transition metal cluster compound according to [5], wherein R³ has 1 to 10 carbon atoms.

[8] The transition metal cluster compound according to any one of [1] to [4], wherein the ligand is a ligand represented by the following general formula (4) or (5):

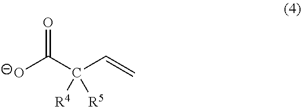

wherein R⁴ and R⁵ are each independently a hydrocarbon group; and

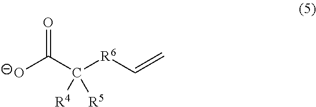

wherein R⁴ and R⁵ are each independently a hydrocarbon group, and R⁶ is a hydrocarbon chain having one or more carbon atoms.

[9] The transition metal cluster compound according to [8], wherein R⁴ and R⁵ each have 1 to 6 carbon atoms.

[10] The transition metal cluster compound according to [8], wherein R⁴ and R⁵ form a ring structure having 1 to 10 carbon atoms.

[11] The transition metal cluster compound according to any one of [8] to [10], wherein R⁶ has 1 to 10 carbon atoms.

[12]A method for producing the transition metal cluster compound according to any one of [1] to [11], wherein a carboxylic acid having a structure represented by the general formula (1) is reacted with a compound containing the transition metal element in a solution.

[13] The method for producing a transition metal cluster compound according to [12], wherein a carboxylic acid having a structure represented by the general formula (1) is mixed into an alcohol solution of a compound containing the transition metal element and reacted in the solution.

[14] The method for producing a transition metal cluster compound according to [12], wherein a carboxylic acid having a structure represented by the general formula (1) is reacted with an alcohol solution of an alkoxide of the transition metal element.

[15]A photosensitive composition comprising the transition metal cluster compound according to any one of [1] to [11].

[16] The photosensitive composition according to [15], wherein the transition metal cluster compound accounts for 50% by mass or more of components of the photosensitive composition excluding a solvent.

[17] The photosensitive composition according to [15] or [16], wherein the photosensitive composition reacts with light having a wavelength of 6.5 to 13.5 nm.

[18]A pattern forming method comprising a step of coating the photosensitive composition according to any one of [15] to [17] onto a substrate, a step of exposing the composition to chemical radiation, and a step of developing the composition using a developer.

[19]A pattern forming method comprising a step of forming a photosensitive layer on a substrate using the photosensitive composition according to any one of [15] to [17], a step of exposing the photosensitive layer to chemical radiation through a photomask, and a step of developing the photosensitive layer using a developer.

[20] The pattern forming method according to [18] or [19], wherein the developer comprises an organic solvent having a solubility parameter (SP value) of 7.5 to 11.

[21]A method for producing a substrate having a patterned layer obtained by the pattern forming method according to [18] or [19].

### Effect of the Invention

The transition metal cluster compound and the photosensitive composition according to the present invention, because they have a property in which a vinyl group terminal is easily polymerized, have a vinyl group reacted with other vinyl groups in a chain reaction for polymerization upon irradiation with absorbed light energy after exposure to extreme ultraviolet (EUV) rays, and become less dissoluble in organic solvents. In the area not exposed to extreme ultraviolet (EUV) rays, a carboxylic acid containing an alkyl or aryl chain that is easily dissolved in an unreacted organic solvent is present in the metal cluster compound, and therefore the area is easily dissolved in the organic solvent. As a result, there is a difference in the dissolution rate between the exposed and unexposed area in the organic solvent, which makes it easy to achieve high development contrast. Therefore, the metal cluster compound composed of a vinyl group that is easily reactive upon irradiation with light energy of extreme ultraviolet (EUV) rays, and a carboxylic acid containing an alkyl or aryl chain that is easily dissolved in an organic solvent, can achieve extremely high exposure sensitivity in extreme ultraviolet (EUV) lithography.

It is also highly sensitive to extreme ultraviolet (EUV) exposure, and can, for example, form a half-pitch (hp) 100 nm L&S (1: 1) pattern with high resolution in an electron beam (EB) lithography test.

### Mode(S) for Carrying Out the Invention

The following is an explanation of the present invention based on embodiments. However, the present invention is not limited to the embodiments.

As used herein, the numerical range in the description where “to” is used as a notation expression to represent the numerical range from the lower limit to the upper limit refers to a numerical range specified as being equal to or more than the lower limit value and equal to or less than the upper limit value, including the lower limit value and the upper limit value. In the present invention, the cluster compound means a metal complex molecule having a metal atom and a ligand, in which a plurality of metal atoms are bonded to each other directly or through a bridging ligand.

### [Transition Metal Cluster Compound]

A transition metal cluster compound according to one embodiment of the present invention (hereinafter also referred to as “the present cluster compound”) is a compound composed of a transition metal element and a ligand represented by the following general formula (1) and forming a cluster centered on the transition metal element. The present cluster compound may contain oxygen and/or hydroxyl groups in the structure, and may preferably contain μ-oxo ligands (μ-O) and/or μ-hydroxy ligands (μ-OH).

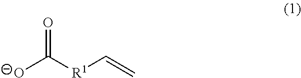

wherein R¹ is a hydrocarbon chain having one or more carbon atoms.

The transition metal element in the present cluster compound is preferably one or more selected from zirconium, hafnium, and titanium, of which zirconium or hafnium is preferred. Hafnium is an element in the same group as zirconium and has very similar chemical and physical properties.

The present cluster compound preferably contains 2 or more and 12 or less transition metal elements, more preferably 4 or more and 12 or less, even more preferably 4 or more and 6 or less, and particularly preferably 6.

In the general formula (1), R¹ is a hydrocarbon chain having 1 or more carbon atoms, preferably a hydrocarbon chain having 1 or more and 20 or less carbon atoms, and more preferably a hydrocarbon chain having 1 or more and 10 or less carbon atoms. In particular, it is preferably a saturated hydrocarbon chain having 1 or more carbon atoms.

The ligand is preferably a ligand represented by the following general formula (2) or (3).

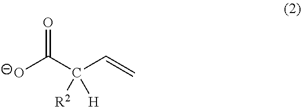

wherein R² is a hydrocarbon group.

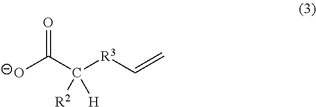

wherein R² is a hydrocarbon group, and R³ is a hydrocarbon chain having one or more carbon atoms.

In the general formula (2) or (3), R² is a hydrocarbon group having 1 or more carbon atoms, preferably a hydrocarbon group having 1 or more and 20 or less carbon atoms, more preferably a hydrocarbon group having 1 or more and 10 or less carbon atoms, and even more preferably a hydrocarbon group having 1 or more and 6 or less carbon atoms. In particular, it is preferably a saturated hydrocarbon group having 1 or more carbon atoms.

R³ is a hydrocarbon chain having 1 or more carbon atoms, preferably a hydrocarbon chain having 1 or more and 20 or less carbon atoms, and more preferably a hydrocarbon chain having 1 or more and 10 or less carbon atoms. In particular, it is preferably a saturated hydrocarbon chain having 1 or more carbon atoms.

When R² or R³ is in the above range, the resulting coating film becomes solid, and thus the resist pattern after development tends to be rectangular.

The ligand is preferably a ligand represented by the following general formula (4) or (5).

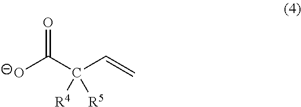

wherein R⁴ and R⁵ are each independently a hydrocarbon group.

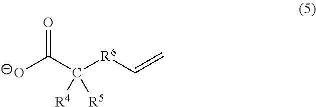

wherein R⁴ and R⁵ are each independently a hydrocarbon group, and R⁶ is a hydrocarbon chain having one or more carbon atoms.

In the general formula (4) or (5), R⁴ and R⁵ are each a hydrocarbon group having 1 or more carbon atoms, preferably a hydrocarbon group having 1 or more and 20 or less carbon atoms, more preferably a hydrocarbon group having 1 or more and 10 or less carbon atoms, and even more preferably a hydrocarbon group having 1 or more and 6 or less carbon atoms. In particular, it is preferably a saturated hydrocarbon group having 1 or more carbon atoms.

R⁴ and R⁵ may be connected to each other to form a ring structure. In this case, it is preferable to form a ring structure having 1 to 10 carbon atoms.

R⁶ is a hydrocarbon chain having 1 or more carbon atoms, preferably a hydrocarbon chain having 1 or more and 20 or less carbon atoms, and more preferably a hydrocarbon chain having 1 or more and 10 or less carbon atoms. In particular, it is preferably a saturated hydrocarbon chain having 1 or more carbon atoms.

When R⁴, R⁵, or R⁶ is in the above range, the resulting coating film becomes solid, and thus the resist pattern after development tends to be rectangular.

Examples of R¹, R³, and R⁶, which are hydrocarbon chains having 1 or more carbon atoms, include methylene, ethylene, trimethylene, tetramethylene, pentamethylene, hexamethylene, heptamethylene, and octamethylene.

Examples of aromatic chains include phenylene, biphenylene, terphenylene, quaterphenylene, and naphthylene.

The structures of the ligands represented by the general formulas (1) to (5) can be analyzed by NMR.

The molecular weight of the present cluster compound is preferably 1,000 to 5,000, more preferably 1,000 to 4,000, and even more preferably 1,500 to 3,000.

When the molecular weight of the present cluster compound is equal to or less than the upper limit, the volume becomes small, and thus a reduction in roughness and an improvement in resolution are expected, while when the molecular weight is equal to or more than the lower limit, the sensitivity and solubility contrast tend to improve. However, the molecular weight described above is only a guide and does not determine the lithographic properties alone.

### [Production Method]

The present cluster compound can be produced, for example, by reacting a carboxylic acid having a structure represented by the general formula (1) with a compound containing a transition metal element in a solution. Preferably, a carboxylic acid having a structure represented by the general formula (1) is mixed into an alcohol solution of a compound containing a transition metal element and reacted in the solution. More preferably, a carboxylic acid having a structure represented by the general formula (1), such as a vinyl alkyl carboxylic acid or a vinyl aryl carboxylic acid, is mixed into an alcohol solution of a transition metal alkoxide and reacted in the solution.

More specifically, a carboxylic acid having a structure represented by the general formula (1) and a solution containing a transition metal element are mixed in a reaction vessel, and the mixture is stirred. A solvent may be added to dissolve the raw materials. The reaction temperature is preferably room temperature to 150° C., more preferably room temperature to 100° C., from the viewpoints of completing the reaction and preventing undesirable side reactions. The reaction time is preferably 1 to 100 hours, more preferably 1 to 24 hours.

If the product precipitates or crystallizes after the reaction, it can be filtered to obtain the present cluster compound. The solution after the reaction may be cooled to −30° C. to 20° C. for the purpose of obtaining a precipitate or crystals of the product.

If no precipitate of the product is observed after the reaction, the product can be reprecipitated by contacting the reaction solution with a poor solvent.

The reaction vessel is preferably a sealed vessel, and for small amounts, for example, a Schlenk tube or the like may be used; and the reaction is preferably performed in a nitrogen or argon atmosphere.

The reaction vessel is preferably a flask equipped with a reflux tube, and when heating, the reaction is preferably performed in a nitrogen or argon atmosphere.

Examples of the compound containing a transition metal element include transition metal alkoxides, transition metal amides, and transition metal chlorides. Among these, transition metal alkoxides are preferred from the viewpoints of ease of handling and reactivity.

Examples of the transition metal alkoxides include zirconium methoxide, zirconium ethoxide, zirconium n-propoxide, zirconium i-propoxide, zirconium n-butoxide, zirconium t-butoxide, zirconium 2-ethylhexoxide, hafnium methoxide, hafnium ethoxide, hafnium n-propoxide, hafnium i-propoxide, hafnium n-butoxide, hafnium t-butoxide, hafnium 2-ethylhexoxide, titanium methoxide, titanium ethoxide, titanium n-propoxide, titanium i-propoxide, titanium n-butoxide, titanium t-butoxide, and titanium 2-ethylhexoxide.

Examples of the vinyl alkyl carboxylic acid in the carboxylic acid having a structure represented by the general formula (1) include 3-butenoic acid, 4-pentenoic acid, 5-hexenoic acid, 6-heptenoic acid, 7-octenoic acid, 8-nonenoic acid, 2-methyl-3-butenoic acid, 2-ethyl-3-butenoic acid, 2-methyl-4-pentenoic acid, 2-ethyl-4-pentenoic acid, 2-methyl-5-hexenoic acid, 2-ethyl-5-hexenoic acid, 2,2-dimethyl-3-butenoic acid, 2,2-dimethyl-4-pentenoic acid, and 2,2-dimethyl-5-hexenoic acid.

Examples of the vinyl aryl carboxylic acid in the carboxylic acid having a structure represented by the general formula (1) include 4-vinylbenzoic acid, 4′-vinylbiphenyl-4-carboxylic acid, and 4-vinyl-1-naphthoic acid.

Examples of the solvent used in the reaction include ester-based solvents, ketone-based solvents, alcohol-based solvents, amide-based solvents, sulfoxide-based solvents, ether-based solvents, and hydrocarbon-based solvents. Among these, alcohol-based solvents are preferred, and an alcohol solvent having 1 to 10 carbon atoms is more preferred. Such solvents are preferably those that dissolve a transition metal compound. In particular, when the compound containing a transition metal element is an alkoxide of a transition metal element, it is preferred that the alkoxide species and the solvent species are the same.

In particular, when the compound containing a transition metal element is an alkoxide of a transition metal element, it is preferable to use an alcohol as the solvent. The concentration of the transition metal alkoxide in such a solution is preferably 50% by weight to 90% by weight, particularly preferably 60% by weight to 80% by weight.

The substance amount ratio of the compound containing a transition metal element to the carboxylic acid having a structure represented by the general formula (1) (such as a vinyl alkyl carboxylic acid or a vinyl aryl carboxylic acid) is preferably 1: 2 to 1: 20, more preferably 1: 3 to 1: 10.

### [Photosensitive Composition]

A photosensitive composition according to one embodiment of the present invention (hereinafter also referred to as “the present photosensitive composition”) contains the present cluster compound. The present photosensitive composition may contain only one type of the present cluster compound, or may contain two or more types thereof.

The content of the present cluster compound in the present photosensitive composition is generally 50% by mass to 100% by mass, preferably 70% by mass to 100% by mass, and particularly preferably 80% by mass to 100% by mass, relative to the total of components of the photosensitive composition excluding a solvent.

When the content of the present cluster compound in the present photosensitive composition is equal to or more than the lower limit, good exposure sensitivity can be achieved.

### [Photoacid Generator]

The present photosensitive composition can also function by containing, together with the present cluster compound, a photoacid generator that generates an acid upon the action of chemical radiation.

Examples of the chemical radiation include emission ray spectra of mercury lamps, far ultraviolet rays represented by excimer lasers, extreme ultraviolet (EUV) rays, X-rays, and electron beams; from the viewpoint of resolution, it is preferred that the exposure wavelength is short, and extreme ultraviolet (EUV) rays emitting light having a wavelength of 6.5 to 13.5 nm are preferred.

The photoacid generator that generates an acid by such chemical radiation is not particularly limited as long as it is a known photoacid generator, and is preferably a compound that generates an organic acid, such as at least one of sulfonic acid, bis(alkylsulfonyl)imide, and tris(alkylsulfonyl)methide, upon irradiation with chemical radiation.

The photoacid generator can be used alone or in combination with two or more types. In the case where two or more types of photoacid generators are used in combination, embodiments such as (1) the use of two types of photoacid generators having different acid strengths in combination, or (2) the use of two types of photoacid generators having different sizes (molecular weight or number of carbon atoms) of generated acid in combination, are preferred.

Examples of the embodiment (1) include the use of a sulfonic acid generator having fluorine and a tris(fluoroalkylsulfonyl)methide acid generator in combination, the use of a sulfonic acid generator having fluorine and a sulfonic acid generator having no fluorine in combination, and the use of an alkyl sulfonic acid generator and an aryl sulfonic acid generator in combination.

Examples of the embodiment (2) include the use of two types of acid generators that generate acid anions with a difference of 4 or more carbon atoms in combination.

In particular, the present photosensitive composition is preferably a photosensitive composition used for chemical radiation, because the development speed in a developer changes if it is a photosensitive composition that reacts with chemical radiation, and a pattern can be formed after a certain time of development.

For chemical radiation, shorter wavelength light is preferred because of the higher resolution that can be achieved, and light having a wavelength of 6.5 to 13.5 nm, i.e., extreme ultraviolet (EUV) rays, is preferred.

In other words, the present photosensitive composition is preferably a photosensitive composition that reacts with light having a wavelength of 6.5 to 13.5 nm.

The present photosensitive compound reacts with light even when used alone in the photosensitive composition. The photoacid generator, when it is added, acts synergistically with the present cluster compound in the photosensitive composition, so that the photosensitivity of the present cluster compound can be increased. Therefore, if a photosensitive composition composed only of the present cluster compound does not have sufficient photosensitivity for the required specifications, it is preferable to add a photoacid generator.

When the present photosensitive composition contains a photoacid generator, the content of the photoacid generator in the present photosensitive composition (the total amount if a plurality of photoacid generators are used in combination) is preferably 0.1% by mass to 30% by mass, more preferably 0.5% by mass to 20% by mass, and even more preferably 1% by mass to 15% by mass, based on the total of the components of the photosensitive composition excluding a solvent.

When the content of the photoacid generator in the photosensitive composition is equal to or more than the lower limit, the effect of increasing the photosensitivity can be achieved, while when the content is equal to or less than the upper limit, good film-forming properties based on the photosensitive compound according to the present invention can be achieved because it is less affected by the poor film-forming properties of the photoacid generator, which are preferred.

### [Solvent]

The present photosensitive composition generally contains a solvent for preparation.

The solvent for preparing the photosensitive composition is not particularly limited as long as it dissolves each component, and examples thereof include alkylene glycol monoalkyl ether carboxylates (such as propylene glycol monomethyl ether acetate (PGMEA; 1-methoxy-2-acetoxypropane)), alkylene glycol monoalkyl ethers (such as propylene glycol monomethyl ether (PGME; 1-methoxy-2-propanol)), alkyl lactate esters (such as ethyl lactate and methyl lactate), cyclic lactones (such as γ-butyrolactone, preferably having 4 to 10 carbon atoms), linear or cyclic ketones (such as 2-heptanone and cyclohexanone, preferably having 4 to 10 carbon atoms), alkylene carbonates (such as ethylene carbonate and propylene carbonate), alkyl carboxylic acid (preferably alkyl acetates such as butyl acetate), alkyl alkoxy acetates (ethyl ethoxypropionate), alkyl amides (N,N-dimethylformamide), and alkyl sulfoxides (dimethyl sulfoxide). Examples of other usable solvents include those described in paragraphs [0244] and following of U.S. Patent Application Publication No. 2008/0248425 A1.

Among these, N,N-dimethylformamide, dimethyl sulfoxide, alkylene glycol monoalkyl ether carboxylates, and alkylene glycol monoalkyl ethers are preferred.

These solvents may be used alone or in combination of two or more types. In the case where two or more types are mixed, it is preferable to mix a solvent having a hydroxyl group with a solvent having no hydroxyl group.

As the solvent having a hydroxyl group, alkylene glycol monoalkyl ethers are preferred; and as the solvent having no hydroxyl group, alkylene glycol monoalkyl ether carboxylate, N,N-dimethylformamide, and dimethyl sulfoxide are preferred.

The content of the solvent in the total amount of the present photosensitive composition can be adjusted as appropriate according to the film thickness of a pattern to be formed or other factors; and in general, the content is adjusted such that the total concentration of the components of the photosensitive composition excluding the solvent is 0.5% by mass to 30% by mass, preferably 1.0% by mass to 20% by mass, more preferably 1.5% by mass to 10% by mass, and even more preferably 1.5% by mass to 5% by mass.

### [Surfactant]

Preferably, the present photosensitive composition further contains a surfactant. As the surfactant, fluorine-based and/or silicon-based surfactants are preferred.

Examples of the surfactants that fall into these categories include MEGAFACE F176 and MEGAFACE R08 manufactured by DIC Corp., PF656 and PF6320 manufactured by OMNOVA, Troysol S-366 manufactured by Troy Chemical Industries, FLUORAD FC430 manufactured by Sumitomo 3M Limited, and Polysiloxane Polymer KP-341 manufactured by Shin-Etsu Chemical Co., Ltd.

In addition, surfactants other than the fluorine-based and/or silicone-based surfactants can be used. Specific examples thereof include polyoxyethylene alkyl ethers and polyoxyethylene alkyl aryl ethers.

Other known surfactants can also be used as appropriate. Examples of other usable surfactants include those described in paragraphs [0273] and following of U.S. Patent Application Publication No. 2008/0248425 A1.

These surfactants may be used alone or in combination of two or more types.

The content of the surfactant is preferably 0.0001% by mass to 2% by mass, more preferably 0.001% by mass to 1% by mass, relative to the total of the components of the photosensitive composition excluding a solvent.

### [Resin]

The present photosensitive composition, although capable of forming a pattern by itself, may also contain a resin material in addition to the present cluster compound. The resin material is not particularly limited as long as it dissolves in a solvent, and examples thereof include novolac resins, styrene resins, and acrylic resins, which may be used alone or in combination of two or more types; and the resin material may contain groups such as a dissolution inhibiting group that decomposes and a crosslinking group that crosslinks, due to chemical active species such as acids and radicals, in the molecular structure, and may be a copolymer resin of two or more types. Examples of the dissolution inhibiting group that decomposes due to chemical active species such as acids and radicals include alkoxycarbonyl groups and acetal groups. Examples of the crosslinking group that crosslinks due to chemical active species such as acids and radicals include vinyl groups, carbodiimide groups, N-hydroxyester groups, imide ester groups, maleimide groups, haloacetyl groups, pyridyl disulfide groups, hydrazide groups, alkoxyamino groups, and diazirine groups.

### [Other Additives]

The present photosensitive composition may appropriately contain, in addition to the components described above, a carboxylic acid, a carboxylic acid onium salt, a dissolution inhibiting compound having a molecular weight of 3,000 or less as described in Proceeding of SPIE, 2724,355 (1996) or the like, a dye, a plasticizer, a photosensitizer, a light absorber, a crosslinking agent, an antioxidant, and the like.

In particular, a carboxylic acid is preferably used for improving performance. Preferred examples of the carboxylic acid include aromatic carboxylic acids such as a benzoic acid and a naphthoic acid.

The content of the carboxylic acid is preferably 0.01% by mass to 10% by mass, more preferably 0.01% by mass to 5% by mass, and even more preferably 0.01% by mass to 3% by mass, relative to the total of the components of the photosensitive composition excluding a solvent.

### [Method for Producing Present Photosensitive Composition]

The present photosensitive composition can be produced by dissolving the present cluster compound, and a photoacid generator and other components, which are used as necessary, in a solvent for preparing a solution, and filtering the solution through a filter as necessary. The filter is preferably a filter made of polytetrafluoroethylene, polyethylene, or nylon having a pore size of 0.2 μm or less, preferably 0.1 μm or less, and more preferably 0.05 μm or less.

### [Pattern Forming Method]

A pattern forming method according to one embodiment of the present invention (hereinafter also referred to as “the present pattern forming method”) includes a step of forming a photosensitive layer on a substrate using the present photosensitive composition, a step of irradiating a predetermined area of the photosensitive layer with chemical radiation to perform pattern exposure, and a step of developing the photosensitive layer after exposure with a developer to selectively remove the exposed or unexposed area of the photosensitive layer.

Further, a pattern forming method according to one embodiment of the present invention may include a step of forming a photosensitive layer on a substrate using the present photosensitive composition, a step of exposing the photosensitive layer to chemical radiation through a photomask, and a step of developing the photosensitive layer using a developer.

### [Step of Forming Photosensitive Layer]

The photosensitive layer can be formed by coating the present photosensitive composition on a substrate (e.g., silicon, silicon dioxide coating) such as that used in the production of integrated circuit elements by a suitable coating method such as a spinner, and then drying at 50° C. to 150° C. The photosensitive layer is preferably in the form of a solid.

In this step, a commercially available inorganic or organic anti-reflective film can be used as necessary. In addition, an anti-reflective film can be coated on a lower layer of the resist.

### [Exposure Step]

In the present invention, the term “exposure” includes not only exposure by far ultraviolet rays represented by mercury lamps and excimer lasers, X-rays, extreme ultraviolet (EUV) rays, and the like, but also lithography by particle beams such as electron beams and ion beams, unless otherwise specified.

The exposure can be performed by irradiating a predetermined area of the formed photosensitive layer with chemical radiation through a predetermined photomask to perform pattern exposure, or by irradiating the area with electron beams to lithograph without using a mask (direct lithography) and to perform pattern exposure.

The chemical radiation is not particularly limited, and examples thereof include KrF excimer lasers, ArF excimer lasers, extreme ultraviolet (EUV) rays, and electron beams, of which extreme ultraviolet (EUV) rays and electron beams are preferred, and as described above, extreme ultraviolet (EUV) rays emitting light having a wavelength of 6.5 to 13.5 nm are preferred.

After the exposure, baking (heating) may or may not be performed prior to development.

When baking (heating) is performed, the heating temperature is preferably 50° C. to 150° C., more preferably 60° C. to 150° C., and even more preferably 80° C. to 140° C.

When baking (heating) is performed, the heating time is preferably 30 to 300 seconds, more preferably 30 to 180 seconds, and even more preferably 30 to 90 seconds.

Heating may be performed by a means provided in a normal exposure/developing machine, or by using a hot plate or the like.

### [Development Step]

After exposure, the photosensitive layer is developed using a developer to selectively remove the exposed or unexposed area.

### <Developer>

As the developer, it is preferable to use an organic solvent, preferably an organic solvent having a vapor pressure of 5 kPa or less at 20° C., more preferably 3 kPa or less, and particularly preferably 2 kPa or less. By adjusting the vapor pressure of the organic solvent to 5 kPa or less, evaporation of the developer on the substrate or in the developing cup is suppressed, and the temperature uniformity within the surface of the pattern-formed substrate is improved, resulting in good dimensional uniformity within the surface of the pattern-formed substrate.

As the organic solvent used as the developer, various organic solvents can be used, and for example, at least one solvent selected from ester-based solvents, ketone-based solvents, alcohol-based solvents, amide-based solvents, sulfoxide-based solvents, ether-based solvents, and hydrocarbon-based solvents can be used.

In particular, it is preferred that the developer contains at least one solvent selected from amide-based solvents, ketone-based solvents, ester-based solvents, alcohol-based solvents, and ether-based solvents.

Examples of the ester-based solvents include alkyl carboxylic acid-based solvents such as methyl acetate, butyl acetate, ethyl acetate, isopropyl acetate, amyl acetate, ethyl-3-ethoxypropionate, propylene glycol diacetate, 3-methoxybutyl acetate, 3-methyl-3-methoxybutyl acetate, methyl formate, ethyl formate, butyl formate, propyl formate, ethyl lactate, butyl lactate, and propyl lactate; and alkylene glycol monoalkyl ether carboxylate-based solvents such as propylene glycol monomethyl ether acetate (PGMEA; also known as 1-methoxy-2-acetoxypropane), ethylene glycol monoethyl ether acetate, diethylene glycol monobutyl ether acetate, diethylene glycol monoethyl ether acetate, and propylene glycol monoethyl ether acetate. Among these, butyl acetate, amyl acetate, ethyl lactate, and propylene glycol monomethyl ether acetate are more preferred.

Examples of the ketone-based solvents include 1-octanone, 2-octanone, 1-nonanone, 2-nonanone, acetone, 4-heptanone, 1-hexanone, 2-hexanone, diisobutyl ketone, cyclopentanone, cyclohexanone, methylcyclohexanone, phenylacetone, methyl ethyl ketone, methyl amyl ketone, methyl isobutyl ketone, acetylacetone, acetonylacetone, ionone, diacetonyl alcohol, acetyl carbinol, acetophenone, methyl naphthyl ketone, isophorone, and propylene carbonate. Among these, alkyl ketone-based solvents, such as methyl isobutyl ketone, methyl amyl ketone, cyclopentanone, and cyclohexanone, are more preferred.

Examples of the alcohol-based solvents include alcohols such as methyl alcohol, ethyl alcohol, n-propyl alcohol including 1-propanol or 2-propanol, isopropyl alcohol, n-butyl alcohol, sec-butyl alcohol, tert-butyl alcohol, isobutyl alcohol, hexyl alcohol such as n-hexyl alcohol, heptyl alcohol such as n-heptyl alcohol, octyl alcohol such as n-octyl alcohol, and decanol such as n-decanol; glycol-based solvents such as ethylene glycol, diethylene glycol, triethylene glycol, 1,2-propylene glycol, 1,3-propylene glycol, 1,2-butylene glycol, 1,3-butylene glycol, and 1,4-butylene glycol; alkylene glycol monoalkyl ether-based solvents such as ethylene glycol monomethyl ether, propylene glycol monomethyl ether (PGME; also known as 1-methoxy-2-propanol), ethylene glycol monoethyl ether, propylene glycol monoethyl ether, diethylene glycol monomethyl ether, and triethylene glycol monoethyl ether; glycol ether-based solvents such as methoxymethyl butanol and propylene glycol dimethyl ether; and phenol-based solvents such as phenol and cresol. Among these, 1-hexanol, 2-hexanol, 1-octanol, 2-ethyl-hexanol, propylene glycol monomethyl ether, and cresol are more preferred.

Examples of the amide-based solvents include N-methyl-2-pyrrolidone, N,N-dimethylacetamide, N,N-dimethylformamide, hexamethylphosphoric triamide, and 1,3-dimethyl-2-imidazolidinone.

Examples of the sulfoxide-based solvents include dimethyl sulfoxide.

Examples of the ether-based solvents include dioxane, tetrahydrofuran, and tetrahydropyran, in addition to the alkylene glycol monoalkyl ether-based solvents and the glycol ether-based solvents described above.

Examples of the hydrocarbon-based solvents include aromatic hydrocarbon solvents such as toluene and xylene; and aliphatic hydrocarbon solvents such as pentane, hexane, octane, decane, and dodecane.

It is preferred that the developer contains one or more solvents selected from alkylene glycol monoalkyl ether carboxylate-based solvents, alkylene glycol monoalkyl ether-based solvents, alkyl carboxylic acid-based solvents, and alkyl ketone-based solvent; and it is more preferred that the developer contains one or more solvents selected from dimethylformamide, dimethyl sulfoxide, tetrahydrofuran, ethylene glycol, methyl alcohol, ethyl alcohol, 1-propanol, and 2-propanol.

As the developer, it is preferable to use a developer containing at least one organic solvent selected from the group consisting of ester-based solvents having no hydroxyl group in the molecule, ketone-based solvents having no hydroxyl group in the molecule, ether-based solvents having no hydroxyl group in the molecule, amide-based solvents, and sulfoxide-based solvents.

The organic solvent used as the developer in the present invention is preferably an organic solvent having a solubility parameter (SP value) of 7.5 to 11. An organic solvent having a solubility parameter of 7.5 or more is preferred because the development speed of the dissolved portion increases, while an organic solvent having a solubility parameter of 11 or less is preferred because the development speed of the pattern-formed portion can be suppressed. The solubility parameter of the organic solvent in the developer is more preferably 8 to 11.

In the present invention, the solubility parameter (SP value) is calculated by the method proposed by Fedors et al. Specifically, it is the value determined by reference to “POLYMER ENGINEERING AND SCIENCE, FEBRUARY, 1974, Vol. 14, No. 2, ROBERTF. FEDORS. (P.147-154)”. The SP value is a physical property value determined by the content of hydrophobic and hydrophilic groups in the molecule, and when a mixed solvent is used, it refers to the value of the mixture.

Examples of the organic solvent that satisfies the above SP value include diethylene glycol monomethyl ether (SP value=10.7), triethylene glycol monomethyl ether (SP value=10.7), ethylene glycol monoisopropyl ether (SP value=10.9), ethylene glycol monobutyl ether (SP value=10.2), diethylene glycol monobutyl ether (SP value=10.0), triethylene glycol monobutyl ether (SP value=10.0), ethylene glycol monoisobutyl ether (SP value=9.1), ethylene glycol monohexyl ether (SP value=9.9), diethylene glycol monohexyl ether (SP value=9.7), diethylene glycol mono 2-ethylhexyl ether (SP value=9.3), ethylene glycol monoallyl ether (SP value=10.8), ethylene glycol monophenyl ether (SP value=10.8), ethylene glycol monobenzyl ether (SP value=10.9), propylene glycol monomethyl ether (SP value=10.0), dipropylene glycol monomethyl ether (SP value=9.7), tripropylene glycol monomethyl ether (SP value=9.4), propylene glycol monopropyl ether (SP value=9.6), dipropylene glycol monopropyl ether (SP value=9.8), propylene glycol monobutyl ether (SP value=9.0), dipropylene glycol monobutyl ether (SP value=9.6), ethylene glycol monomethyl ether acetate (SP value=10.0), ethylene glycol monoethyl ether acetate (SP value=9.6), ethylene glycol monobutyl ether acetate (SP value=8.9), diethylene glycol monoethyl ether acetate (SP value=9.4), diethylene glycol monobutyl ether acetate (SP value=9.0), propylene glycol monomethyl ether acetate (SP value=9.4), propylene glycol monoethyl ether acetate (SP value=9.0), and dipropylene glycol monomethyl ether acetate (SP value=9.2).

The organic solvents described above may be used in combination, or may be used in combination with a solvent other than the above or with water.

The concentration of the organic solvent (the total if a plurality of organic solvents are mixed) in the developer is preferably 50% by mass or more, more preferably 70% by mass or more, and even more preferably 90% by mass or more. It is particularly preferred that the developer is substantially composed only of organic solvents. The term “substantially composed only of organic solvents” includes cases where trace amounts of surfactants, antioxidants, stabilizers, defoaming agents, and the like are contained.

The water content in the developer is preferably 10% by mass or less, more preferably 5% by mass or less, and particularly preferably 3% by mass or less; and it is most preferred that the developer contains substantially no water. By adjusting the water content to 10% by mass or less, good development characteristics can be achieved.

An appropriate amount of a surfactant may be added to the developer used in the present invention, as necessary.

As the surfactant, the same surfactants as those described above used in the photosensitive composition according to the present invention may be used.

The amount of the surfactant used is generally 0.001% by mass to 5% by mass, preferably 0.005% by mass to 2% by mass, and more preferably 0.01% by mass to 0.5% by mass, relative to the total amount of the developer.

### <Development Method>

Examples of the development method that can be applied include a method of immersing a substrate into a tank filled with a developer for a certain period of time (dip method), a method of piling up a developer on the surface of a substrate by surface tension and leaving it there for a certain period of time (paddle method), a method of spraying a developer on the surface of a substrate (spray method), and a method of continuously dispensing a developer while scanning a developer dispensing nozzle at a constant speed onto a substrate rotating at a constant speed (dynamic dispense method).

After the development step, a step of stopping the development while replacing the solvent with other solvents may be performed.

The development time is preferably a time required for the present cluster compound and the like in the unexposed or exposed area of the photosensitive layer to be sufficiently dissolved, and is generally preferably 10 to 300 seconds, more preferably 20 to 120 seconds.

The temperature of the developer is preferably 0° C. to 50° C., more preferably 15° C. to 35° C.

The amount of the developer can be adjusted as appropriate according to the development method.

### [Rinsing Step]

The present pattern forming method may include a step of washing using a rinse solution containing an organic solvent after the development step.

### <Rinse Solution>

The organic solvent used in the rinse solution preferably has a vapor pressure of 0.05 kPa or more and 5 kPa or less at 20° C., more preferably 0.1 kPa or more and 5 kPa or less, and most preferably 0.12 kPa or more and 3 kPa or less. By adjusting the vapor pressure of the organic solvent used in the rinse solution to 0.05 kPa or more and 5 kPa or less, the temperature uniformity within the wafer surface is improved, and swelling caused by the penetration of the rinse solution is suppressed, thereby improving the dimensional uniformity within the wafer surface.

As the rinse solution, various organic solvents can be used, and for the present cluster compound, a rinse solution containing at least one organic solvent selected from hydrocarbon-based solvents, ketone-based solvents, ester-based solvents, alcohol-based solvents, amide-based solvents, and ether-based solvents, or water is preferably used.

More preferably, after the development, a step of washing is performed using a rinse solution containing at least one organic solvent selected from ketone-based solvents, ester-based solvents, alcohol-based solvents, amide-based solvents, and hydrocarbon-based solvents. Even more preferably, after the development, a step of washing is performed using a rinse solution containing at least one organic solvent selected from the group consisting of alcohol-based solvents and hydrocarbon-based solvents.

Specific examples of the ketone-based solvents, ester-based solvents, alcohol-based solvents, amide-based solvents, ether-based solvents, and hydrocarbon-based solvents used as the rinse solution are the same as those described above for the developer.

Most preferably, a rinse solution containing at least one organic solvent selected from the group consisting of monohydric alcohol-based solvents, hydrocarbon-based solvents, and amide-based solvents is used.

Examples of the monohydric alcohol-based solvents used in the rinsing step after development include linear, branched, and cyclic monohydric alcohols, and specific examples thereof include 1-butanol, 2-butanol, 3-methyl-1-butanol, tert-butyl alcohol, isopropyl alcohol, cyclopentanol, and cyclohexanol, of which 1-butanol, 2-butanol, 3-methyl-1-butanol, and isopropyl alcohol are preferred.

Examples of the hydrocarbon-based solvents include aromatic hydrocarbon-based solvents such as toluene and xylene, and aliphatic hydrocarbon-based solvents such as octane, decane, and dodecane.

Examples of the amide-based solvents include N,N-dimethylformamide.

The components described above may be used in combination in plural numbers, or may be used in combination with organic solvents other than those described above.

The organic solvent may be mixed with water. However, the water content in the rinse solution is generally 30% by mass or less, preferably 10% by mass or less, more preferably 5% by mass or less, and particularly preferably 3% by mass or less. It is most preferred that the rinse solution contains no water. By adjusting the water content to 30% by mass or less, good development characteristics can be achieved.

An appropriate amount of a surfactant may be added to the rinse solution.

As the surfactant, the same surfactants as those used in the photosensitive composition described above can be used; and the amount of the surfactant used is generally 0.001% by mass to 5% by mass, preferably 0.005% by mass to 2% by mass, and more preferably 0.01% by mass to 0.5% by mass, relative to the total amount of the rinse solution.

### <Rinsing Method>

In the rinsing step, the developed pattern-formed substrate is washed using the rinse solution containing organic solvents described above.

The washing method is not particularly limited, and examples of the method that can be applied include a method of continuously coating a rinse solution onto a substrate rotating at a constant speed (spin coating method), a method of immersing a substrate into a tank filled with a rinse solution for a certain period of time (dip method), and a method of spraying a rinse solution onto the surface of a substrate (spray method). Among these, it is preferable to perform washing by the spin coating method, and then to spin the substrate at a number of rotations of 2,000 to 4,000 rpm after the washing to remove the rinse solution from the substrate. The substrate rotation time can be set within a range where the rinse solution is removed from the substrate according to the number of rotations, and is generally 10 seconds to 3 minutes. It is preferable to perform rinsing at room temperature.

The rinsing time is preferably such that no developing solvent remains on the substrate, and is generally preferably 10 to 300 seconds. It is more preferably 20 to 120 seconds.

The temperature of the rinse solution is preferably 0° C. to 50° C., more preferably 15° C. to 35° C.

The amount of the rinse solution can be adjusted as appropriate according to the rinsing method.

### [Post-Treatment Step]

After developing or rinsing, a treatment may be performed in which the developer or rinse solution adhering on the pattern is removed by using a supercritical fluid.

In addition, after development, rinsing, or supercritical fluid treatment, a heat treatment may be performed to remove the solvent remaining on the pattern. The heating temperature and time are not particularly limited as long as a good resist pattern is obtained, and are generally 40° C. to 160° C. for 10 seconds to 3 minutes. The heat treatment may be performed several times.

### [Applications]

The present photosensitive composition and the present pattern forming method are suitable for use in the production of semiconductor fine circuits, such as the production of ultra-LSIs and high-capacity microchips. In the production of semiconductor fine circuits, a pattern-formed resist film is subjected to circuit formation and etching, and the remaining resist film portion is finally removed using a solvent or the like, so that, unlike so-called permanent resists used in printed circuit boards or the like, no resist film derived from the present photosensitive composition remains on the final product such as a microchip.

### EXAMPLES

Examples of the present invention are described below. However, the present invention is not limited to the following Examples. The terms “parts” and “%” in Examples are on a mass basis unless otherwise specified.

The following compounds 1 to 5 were prepared and obtained as Examples 1 to 5, respectively. The composition and ligand structure of each compound were measured by ¹H-NMR.

The compounds 1 to 2 and 4 to 5 were confirmed as cluster compounds by electrospray ionization mass spectrometry (ESI-MS).

For the ESI-MS measurement, Xevo G2-XS QTof, manufactured by Waters Corp., was used.

The compounds 1 to 2 and 4 to 5 were each dissolved in a mixed solvent of THF and acetonitrile, and filtered through a 0.2 μm filter before use.

For the moving bed, a mixed solvent of THF and acetonitrile was used.

### <Compound 1>

In a reaction vessel, 6.9 mL (21.9 mmol) of a zirconium propoxide solution (70% propanol solution) manufactured by Tokyo Chemical Industry Co., Ltd., 10 mL of 1-propanol (super dehydrated) manufactured by Fujifilm Wako Pure Chemical Corp., and 5.3 g (46.1 mmol) of 5-hexenoic acid manufactured by Tokyo Chemical Industry Co., Ltd. were mixed, the mixture was stirred at room temperature for 1 hour, and then the solvent was removed under reduced pressure to prepare 4.9 g of a yellow oily substance. The resulting substance was analyzed by NMR and found to have a structure represented by the following chemical formula (6).

¹H-NMR (CDCl₃, ppm):5.8-5.6 (br, 1H), 5.1-4.8 (br, 2H), 2.5-1.8 (4H), 1.8-1.4 (br, 2H)

ESI-MS (negative): Cluster-derived peaks at m/z 1600-2100

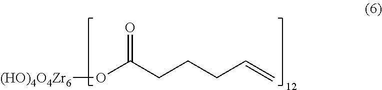

### <Compound 2>

In a reaction vessel, 5.1 mL (16.1 mmol) of a zirconium propoxide solution (70% propanol solution) manufactured by Tokyo Chemical Industry Co., Ltd., 10 mL of 1-propanol (super dehydrated) manufactured by Fujifilm Wako Pure Chemical Corp., and 5.3 g (33.8 mmol) of 8-nonenoic acid manufactured by Tokyo Chemical Industry Co., Ltd. were mixed, the mixture was stirred at room temperature for 1 hour, and then the solvent was removed under reduced pressure to prepare 5.9 g of a yellow oily substance. The resulting substance was analyzed by NMR and found to have a structure represented by the following chemical formula (7).
¹H-NMR (CDCl₃,ppm):5.9-5.7 (m, 1H), 5.1-4.9 (m, 2H), 2.3-1.8 (br, 4H), 1.8-1.0 (br, 6H)

ESI-MS (negative): Cluster-derived peaks at m/z 1500-2500

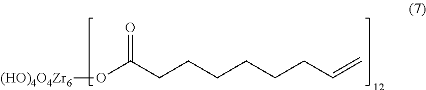

### <Compound 3>

In a reaction vessel, 5.0 mL (15.9 mmol) of a zirconium propoxide solution (70% propanol solution) manufactured by Tokyo Chemical Industry Co., Ltd., 30 mL of 1-propanol (super dehydrated) manufactured by Fujifilm Wako Pure Chemical Corp., and 5.0 g (33.8 mmol) of 4-vinylbenzoic acid manufactured by Tokyo Chemical Industry Co., Ltd. were mixed, the mixture was stirred at room temperature for 1 hour, and then the solvent was removed under reduced pressure to prepare 8.0 g of a white solid.

The resulting solid was analyzed by ¹H-NMR and found to contain approximately the same amount of 1-propanol as the 4-vinylbenzoate ligand. The resulting solid was analyzed by NMR and found to have a structure represented by the following chemical formula (8).

¹H NMR (CDCl₃,ppm):8.6-7.6 (br, 2H), 7.5-7.2 (br, 1H), 7.2-6.1 (br, 2H), 6.0-5.6 (br, 1H), 5.5-4.8 (br, 1H)

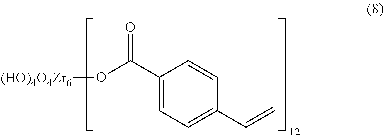

### <Compound 4>

In a reaction vessel, 12.0 g (25.6 mmol) of a zirconium propoxide solution (70% propanol solution) manufactured by Tokyo Chemical Industry Co., Ltd., 10 mL of hexane manufactured by Fujifilm Wako Pure Chemical Corp., and 10.3 g (103 mmol) of 2-methyl-3-butene manufactured by Tokyo Chemical Industry Co., Ltd. were mixed, and the mixture was heated to reflux in an oil bath at 90° C. for 1 hour. The solvent was removed under reduced pressure, the mixture was liquid-separated with hexane and acetonitrile, and the acetonitrile layer was collected and concentrated to prepare an oily residue. The resulting residue was dried to obtain a powder. The resulting solid was analyzed by NMR and found to have a structure represented by the following chemical formula (9).

¹H NMR (CDCl₃,ppm):6.0-5.8 (m, 1H), 5.2-4.8 (m, 2H), 3.2-2.8 (br, 1H), 1.4-1.0 (br, 3H)

ESI-MS (negative): Cluster-derived peaks at m/z 1600-2000

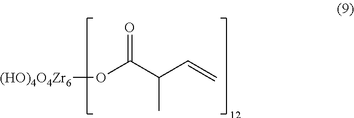

### <Compound 5>

In a reaction vessel, 12.0 g (25.6 mmol) of a zirconium propoxide solution (70% propanol solution) manufactured by Tokyo Chemical Industry Co., Ltd., 10 mL of hexane manufactured by Fujifilm Wako Pure Chemical Corp., and 13.1 g (103 mmol) of 2,2-dimethyl-4-pentenoic acid manufactured by Tokyo Chemical Industry Co., Ltd. were mixed, and the mixture was heated to reflux in an oil bath at 90° C. for 1 hour. The solvent was removed under reduced pressure, the mixture was liquid-separated with hexane and acetonitrile, and the acetonitrile layer was collected and concentrated to prepare an oily residue. The resulting residue was dried to obtain a powder. The resulting solid was analyzed by NMR and found to have a structure represented by the following chemical formula (10).

¹H NMR (CDCl₃,ppm):6.0-5.6 (br, 1H), 5.2-4.9 (m, 2H), 2.5-2.0 (m, 2H), 1.3-1.0 (m, 6H)

ESI-MS (negative): Cluster-derived peaks at m/z 1700-2300

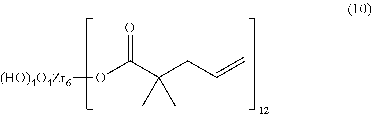

### <Photosensitive Composition (Resist Solution) Preparation>

Each of the compounds 1 to 5 was dissolved in propylene glycol monomethyl ether acetate (referred to as PGMEA) or 2-heptanone at a concentration of 5% by mass as shown in Table 1, and filtered through a 0.2 μm filter to prepare a resist solution.

### <Resist Film Formation and Patterning>

The prepared resist solution was spin-coated onto a pattern-formed substrate (silicon wafer) to form a resist film having a thickness of approximately 40 nm. The resulting resist film was baked at 90° C. for 90 seconds, and then patterned using an electron beam lithography system (electron beam acceleration voltage: 100 keV).

### <Development>

After patterning, the compounds 1 to 5 were developed (25° C., 60 seconds) with n-butyl acetate (may be referred to as nBA) or propylene glycol monomethyl ether acetate (PGMEA) as shown in Table 1 to obtain negative-type patterns, which were designated as Examples 1 to 5, respectively.

### <Sensitivity Measurement>

Extreme ultraviolet (EUV) exposure was performed at different doses of irradiation, and the film thickness of each developed pattern was measured using a contact step gauge. The dose of electron beam irradiation at which the increase in film thickness was maximized was determined as the sensitivity. The sensitivity is higher when the exposure dose (unit: mJ/cm²) is smaller, and is lower when the exposure dose is larger.

The results are shown in Table 1.

### Comparative Example 1

Polyhydroxystyrene (PHS, weight average molecular weight of 4,000) manufactured by Sigma-Aldrich was dissolved in propylene glycol monomethyl ether acetate at a concentration of 2% by mass, and the solution was filtered through a 0.2 μm filter to prepare a resist solution.

The resist film formation, patterning, development, and sensitivity measurement were performed by the methods described above.

### Comparative Example 2

The Zr₆O₄(OH)₄(methacrylate)₁₂ cluster shown in Reference 1 below (hereinafter referred to as Compound 6, see the following chemical formula (11)) used as a resist solution, in which the sensitivity was approximately 25 mJ/cm², was designated as Comparative Example 2 (see, for example, FIG. 6 in Reference 1).

### REFERENCE 1

Sho Kataoka etc., “Enhanced Solubility of Zirconium Oxo Clusters from Diacetoxyzirconium(IV) Oxide Aqueous Solution as Inorganic Extreme-Ultraviolet Photoresists”, Eur.J.Inorg.Chem. 2022, e202200050

[URL: https://doi.org/10.1002/ejic.202200050]

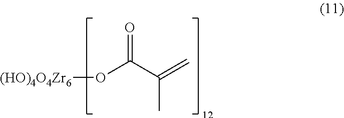

The resist solutions in Comparative Examples 1 and 2 were evaluated in the same manner as in Examples 1 to 5. The results are shown in Table 1.

TABLE 1

| | | | | Sensitivity |
|---|---|---|---|---|
| | Compound | Resist solvent | Developer | (mJ/cm²) |
| Example 1 | Compound 1 | PGMEA | nBA | 1 |
| Example 2 | Compound 2 | PGMEA | nBA | 1 |
| Example 3 | Compound 3 | PGMEA | nBA | 8 |
| Example 4 | Compound 4 | 2-Heptanone | PGMEA | 20 |
| Example 5 | Compound 5 | 2-Heptanone | PGMEA | 15 |
| Comparative | PHS | PGMEA | nBA | >80 |
| Example 1 | | | | |
| Comparative | Compound 6 | PGMEA | nBA | 25 |
| Example 2 | | | | |

Based on the results in Table 1, it was confirmed that the sensitivity in Examples 1 to 5 was higher than that in Comparative Example 1 or 2, and that the sensitivity in Examples 1 and 2 was particularly high.

### <Resolution Evaluation>

The photosensitive compositions in Examples 1 to 5 and Comparative Example 1 were evaluated for resolution using an electron beam lithography system. The resolution was evaluated by patterning a line-and-space (line: space=1:1) pattern with a half-pitch (hp) of 100 nm, developing the pattern, and observing the pattern using a scanning electron microscope. It was confirmed that a line-and-space pattern of hp 100 nm was observed, allowing a fine pattern to be formed.

Based on the above results, it is found that the photosensitive composition according to the present invention is capable of forming a fine pattern and exhibits extremely high sensitivity to extreme ultraviolet (EUV) rays. In contrast, in Comparative Example 1, although a fine pattern can be formed, there is room for improvement in sensitivity.

The invention claimed is:

1. A transition metal cluster compound, comprising:
a transition metal element, and
a ligand represented by the following general formula (1), the transition metal cluster compound forming a cluster centered on the transition metal element: 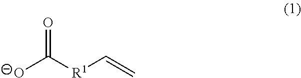
wherein R¹ is a hydrocarbon chain having one or more carbon atoms.

2. The transition metal cluster compound according to claim 1, wherein the transition metal element comprises at least one element selected from the group consisting of zirconium, hafnium, and titanium.

3. The transition metal cluster compound according to claim 1, comprising 2 or more and 12 or less of the transition metal elements and a ligand represented by the general formula (1).

4. The transition metal cluster compound according to claim 1, wherein R¹ is a saturated hydrocarbon chain having one or more carbon atoms.

5. The transition metal cluster compound according to claim 1, wherein the ligand is a ligand represented by the following general formula (2) or (3): 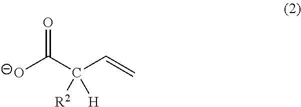
wherein R² is a hydrocarbon group; and 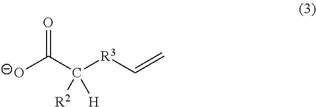
wherein R² is a hydrocarbon group, and R³ is a hydrocarbon chain having one or more carbon atoms.

6. The transition metal cluster compound according to claim 5, wherein R² has 1 to 6 carbon atoms.

7. The transition metal cluster compound according to claim 5, wherein R³ has 1 to 10 carbon atoms.

8. The transition metal cluster compound according to claim 4, wherein the ligand is a ligand represented by the following general formula (4) or (5): 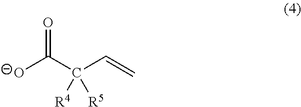
wherein R⁴ and R⁵ are each independently a hydrocarbon group; and 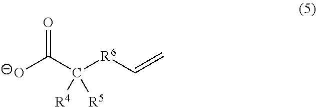
wherein R⁴ and R⁵ are each independently a hydrocarbon group, and R⁶ is a hydrocarbon chain having one or more carbon atoms.

9. The transition metal cluster compound according to claim 8, wherein R⁴ and R⁵ each have 1 to 6 carbon atoms.

10. The transition metal cluster compound according to claim 8, wherein R⁴ and R⁵ form a ring structure having 1 to 10 carbon atoms.

11. The transition metal cluster compound according to claim 8, wherein R⁶ has 1 to 10 carbon atoms.

12. A method for producing the transition metal cluster compound according to claim 1, comprising:
reacting a carboxylic acid having a structure represented by the general formula (1) with a compound containing the transition metal element in a solution.

13. The method for producing a transition metal cluster compound according to claim 12, comprising:
mixing a carboxylic acid having a structure represented by the general formula (1) into an alcohol solution of a compound containing the transition metal element, and
reacting the carboxylic acid having a structure represented by the general formula (1) in the solution.

14. The method for producing a transition metal cluster compound according to claim 12, comprising:
reacting a carboxylic acid having a structure represented by the general formula (1) with an alcohol solution of an alkoxide of the transition metal element.

15. A photosensitive composition, comprising:
the transition metal cluster compound according to claim 1.

16. The photosensitive composition according to claim 15, wherein the transition metal cluster compound accounts for 50% by mass or more of components of the photosensitive composition excluding a solvent.

17. The photosensitive composition according to claim 15, wherein the photosensitive composition reacts with light having a wavelength of 6.5 to 13.5 nm.

18. A pattern forming method, comprising:
coating the photosensitive composition according to claim 15 onto a substrate,
exposing the photosensitive composition to chemical radiation, and
developing the photosensitive composition by a developer.

19. A pattern forming method, comprising:
forming a photosensitive layer on a substrate by the photosensitive composition according to claim 15,
exposing the photosensitive layer to chemical radiation through a photomask, and
developing the photosensitive layer by a developer.

20. The pattern forming method according to claim 18, wherein the developer comprises an organic solvent having a solubility parameter (SP value) of 7.5 to 11.

21. A method, comprising:
producing a substrate having a patterned layer obtained by the pattern forming method according to claim 18.

22. The transition metal cluster compound according to claim 1, consisting of:
a transition metal element, and
a ligand represented by the following general formula (1), the transition metal cluster compound forming a cluster centered on the transition metal element: 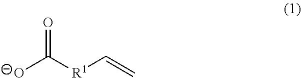
wherein R¹ is a hydrocarbon chain having one or more carbon atoms.
